(19) United States
(12) Patent Application Publication
(10) Pub. No.: US 2024/0410870 A1
(43) Pub. Date: Dec. 12, 2024

(54) MULTI-FACTOR COUPLING COOPERATIVE EARLY WARNING METHOD AND SYSTEM FOR FATIGUE CRACK PROPAGATION OF STEEL STRUCTURE

(71) Applicant: Qingdao University of Technology, Qingdao City (CN)

(72) Inventors: Liming Zhang, Qingdao City (CN); Tao Lan, Chaoyang District (CN); Faxing Zhang, Qingdao City (CN); Zhongyuan Liu, Qingdao City (CN); Juan Lan, Jinan City (CN)

(21) Appl. No.: 18/595,510

(22) Filed: Mar. 5, 2024

(30) Foreign Application Priority Data
Jun. 8, 2023 (CN) 202310671063.9

(51) Int. Cl. G01N 33/2045; G06N 5/01; G06N 5/048

(52) U.S. Cl. CPC G01N 33/2045; G06N 5/013; G06N 5/048

(57) ABSTRACT

The present disclosure relates to a multi-factor coupling cooperative early warning method and system for fatigue crack propagation of a steel structure. The multi-factor coupling cooperative early warning method includes: obtaining multi-physical field monitoring data of a dangerous source distribution point of a steel structure project, and obtaining a monitoring time series data set; establishing an intuitionistic fuzzy matrix of the monitoring time series data set; obtaining uncertainties of indexes by using a grey relation coefficient between the monitoring indexes of physical fields; taking obtained uncertainties as basic probability assignments of evidences; preprocessing the evidences through weighed averaging, and obtaining corrected basic probability assignments; obtaining basic probability assignments of fatigue crack propagation of the steel structure at different development stages; and determining a fatigue crack propagation grade of the dangerous source distribution point of the steel structure project through the basis probability assignment.

### CROSS REFERENCE TO RELATED APPLICATION

This patent application claims the benefit and priority of Chinese Patent Application No. 202310671063.9, filed with the China National Intellectual Property Administration on Jun. 8, 2023, the disclosure of which is incorporated by reference herein in its entirety as part of the present application.

### TECHNICAL FIELD

The present disclosure relates to the technical field of structural monitoring, in particular to the technical field of steel structure project monitoring and early warning, and specifically to a multi-factor coupling cooperative early warning method and system for fatigue crack propagation of a steel structure.

### BACKGROUND

The steel structures are likely to suffer fatigue cracks induced by internal fatigue damage due to various complex loads during use. As a result, their performance can be deteriorated, and a failure and even a catastrophic accident can be caused. In view of that, it is essential to monitor, evaluate and give an early warning of fatigue damage of the steel structures.

Since the fatigue damage of the steel structures is associated with various factors, a single monitoring or detection means cannot ensure the reliability of safety state evaluation results of the steel structures. In addition, monitoring data are still underexplored since monitoring of the steel structures generally highlights monitoring and minimizes analysis at present.

To this end, it is urgent to provide an effective multi-factor coupling evaluation method for fatigue damage of a steel structure, which can identify different development stages of fatigue crack propagation in a steel structure project, and implement time-varying prediction, a stage-based early warning and a probability early warning of a safety state of the steel structure project.

### SUMMARY

In order to solve the shortcomings in the prior art, a main objective of the present disclosure is to provide a multi-factor coupling cooperative early warning method and system for fatigue crack propagation of a steel structure, so as to correctly evaluate a fatigue damage degree and a safety state of a steel structure project and solve one or more problems in the prior art. A technical solution of the present disclosure is as follows:

The present disclosure provides a multi-factor coupling cooperative early warning method for fatigue crack propagation of a steel structure at first. The multi-factor coupling cooperative early warning method includes:
S01, obtaining multi-physical field sensor monitoring data of a dangerous source distribution point of a steel structure project, preprocessing the data, and obtaining a multi-physical field monitoring time series data set;
S02, establishing an intuitionistic fuzzy matrix of the multi-physical field monitoring time series data set based on an interval-valued intuitionistic fuzzy decision-making theory of a grey system theory;
S03, converting the intuitionistic fuzzy matrix into a score function matrix by using a score function, obtaining a grey relation coefficient between monitoring indexes of physical fields, and obtaining uncertainties of the indexes;
S04, introducing a Dempster-Shafer (D-S) evidence theory, taking the uncertainties of the indexes as bases for basic probability assignments of evidences in the D-S evidence theory, and obtaining the basic probability assignments of the evidences;
S05, according to the basic probability assignments of the evidences, introducing a Minkowski distance, establishing a support degree matrix, determining a belief factor, taking the belief factor as a weight for distributing the evidences, performing weighed averaging, and obtaining corrected basic probability assignments;
S06, improving a combination rule of the D-S evidence theory based on a principle of local conflict distribution, fusing the corrected basic probability assignments by using an improved combination rule of the D-S evidence theory, and obtaining basic probability assignments of fatigue crack propagation of the steel structure at different development stages; and
S07, determining a fatigue crack propagation grade of the dangerous source distribution point of the steel structure project by a decision-making method for the basic probability assignment, and implementing time-varying prediction, a stage-based early warning and a probability early warning during the fatigue crack propagation of the steel structure.

In some embodiments, the obtaining multi-physical field sensor monitoring data of a dangerous source distribution point of a steel structure project, preprocessing the data, and obtaining a multi-physical field monitoring time series data set in S01 specifically include:
obtaining multi-sensor real-time monitoring data of the dangerous source distribution point of the steel structure project, where the multi-physical field sensor monitoring data are real-time monitoring data collected by two or more sensors among a strain sensor, a displacement sensor, a stress sensor, a wave velocity sensor, a temperature sensor, an acoustic emission sensor and an electromagnetic radiation sensor according to a time series; and
multi-physical field monitoring time series data are real-time monitoring data of a combination of two or more of monitoring indexes that include a displacement, a strain, a stress, a wave velocity, an osmotic pressure, a temperature, acoustic emission and electromagnetic radiation.

In some embodiments, the establishing an intuitionistic fuzzy matrix of the multi-physical field monitoring time series data set in S02 specifically includes:
S021, obtaining an interval-valued intuitionistic fuzzy number according to the multi-physical field monitoring time series data set and by an interval-valued intuitionistic fuzzy decision-making method based on the grey system theory:

$${e}_{ij} = 〈 u ( x ) , v ( x ) 〉 ; \qquad ( 1 )$$

where u(x) and v(x) denote a membership degree and a non-membership degree of an element x, belonging to a fatigue crack development stage of the steel structure, in a monitoring index μⱼ respectively, j=1, 2, . . . , m; i=1, 2, . . . , n; and
S022, establishing the intuitionistic fuzzy matrix, and establishing an intuitionistic fuzzy decision-making matrix according to a monitoring index and a development stage of the fatigue crack propagation of the steel structure;

$$E = {( {e}_{ij} )}_{m \times n} , \qquad ( 2 )$$

where eᵢⱼ denotes an attribute value under the monitoring index μⱼ of the fatigue crack development stage of the steel structure, and is referred to as the interval-valued intuitionistic fuzzy number.

In some embodiments, the converting the intuitionistic fuzzy matrix into a score function matrix by using a score function, obtaining a grey relation coefficient between monitoring indexes of physical fields, and obtaining uncertainties of the indexes in S03 specifically include:
S031, defining the score function:

$$g ( x ) = u ( x ) - v ( x ) ; \qquad ( 3 )$$

where g(x)∈[−1,1], g(x) expresses a difference between a support degree and an opposition degree, and denotes a net support degree, g(x)=−1 indicates absolute opposition, and g(x)=1 indicates absolute support;
S032, obtaining a score matrix according to the score function:

$$G = {( {g}_{ij} )}_{m \times n} ; \qquad ( 4 )$$

S033, computing the grey relation coefficient between the indexes according to the score matrix:

$${r}_{ij} = \frac{min \vert {g}_{ij} - \overline{{g}_{i}} \vert + ρ max \vert {g}_{ij} - \overline{{g}_{i}} \vert}{\vert {g}_{ij} - \overline{{g}_{i}} \vert + ρ max \vert {g}_{ij} - \overline{{g}_{i}} \vert} , i = 1 , 2 , … , j = 1 , 2 , … ; \qquad ( 5 )$$

where rᵢⱼ denotes the grey relation coefficient between the indexes, gᵢⱼ denotes a score function value, g̅ᵢ denotes an average score function value and ρ denotes a weight coefficient; and
S034, obtaining the uncertainty of the monitoring index by using the grey relation coefficient;

$$DOI ( {μ}_{j} ) = {\frac{1}{m} [ {\sum }_{i = 1}^{m} {( {r}_{ij} )}^{q} ]}^{\frac{1}{q}} ; \qquad ( 6 )$$

where μⱼ denotes the monitoring index, m denotes a number of the monitoring indexes, rᵢⱼ denotes the grey relation coefficient between the indexes, and q denotes a distance measurement coefficient.

In some embodiments, the obtaining basic probability assignments of the monitoring indexes at different development stages by using the uncertainty DOI(μⱼ) in S04 includes:

$${m}_{j}^{*} ( i ) = \frac{( 1 - DOI ( {μ}_{j} ) ) \cdot {r}_{ij}}{{\sum }_{i = 1}^{m} {r}_{ij}} ; \qquad ( 7 )$$

correcting m*ⱼ(i):

$${m}_{j} ( i ) = \frac{{m}_{j}^{*} ( i )}{{\sum }_{i = 1}^{m} {m}_{j}^{*} ( i )} ; \qquad ( 8 )$$

where an obtained mⱼ(i) denotes the basic probability assignment at different development stages under the monitoring index μⱼ.

In some embodiments, the introducing a Minkowski distance, establishing a support degree matrix, determining a belief factor, taking the belief factor as a weight for distributing the evidences, performing weighed averaging, performing weighted averaging, and obtaining a corrected basic probability assignment in S05 specifically include:

S051, defining a Minkowski distance between the evidences, where a Minkowski distance between n-dimensional vectors a(x₁₁, x₁₂ . . . , x₁ₙ) and b(x₂₁, x₂₂ . . . x₂ₙ) is expressed as follows:

$${d}_{12} = \sqrt[p]{{\sum }_{k = 1}^{n} {( \vert {x}_{1 k} - {x}_{2 k} \vert )}^{p}} ; \qquad ( 9 )$$

where a and b are two n-dimensional vectors, x₁ₖ denotes a value of a vector at a first row and a k^{th} column, x₂ₖ denotes a value of a vector at a second row and a k^{th} column, P denotes a Minkowski index, p≥1 and PN⊂N;
S052, computing the Minkowski distance between the evidences by using formula (9) and obtaining a distance matrix dᵢⱼ as follows:

$${d}_{ij} = [ \begin{matrix}0 & & {d}_{12} & & {d}_{1 n} \\ & & & ⋯ & \\ {d}_{21} & & 0 & & {d}_{2 n} \\ & ⋮ & & ⋱ & ⋮ \\ {d}_{n 1} & & {d}_{n 2} & ⋯ & 0\end{matrix} ] ; \qquad ( 10 )$$

S053, quantitatively characterizing a support degree between the evidences through the distance matrix, and defining the support degree supᵢⱼ between the evidences as follows:

$${sup}_{ij} = {e}^{- {d}_{ij}} ; \qquad ( 11 )$$

S054, obtaining the support degree matrix S according to the support degree:

$$S = [ \begin{matrix}1 & {sup}_{12} & {sup}_{13} & & {sup}_{1 n} \\ & & & ⋯ & \\ {sup}_{21} & 1 & {sup}_{23} & & {sup}_{2 n} \\ & ⋮ & & ⋱ & ⋮ \\ {sup}_{n 1} & {sup}_{n 2} & {sup}_{n 3} & ⋯ & 1\end{matrix} ] ; \qquad ( 12 )$$

S055, summing all elements except a designated element in each row of the support degree matrix and obtaining an inter-evidence support degree recᵢ:

$${rec}_{i} = {\sum }_{j = 1 , i \neq j}^{n} {sup}_{ij} , i , j = 1 , 2 , … , n ; \qquad ( 13 )$$

S056, measuring the support degree between the evidences by using the belief factor, where the belief factor δᵢ between the evidences is as follows:

$${δ}_{i} = \frac{{rec}_{i}}{{\sum }_{i = 1}^{n} {rec}_{i}} , i = 1 , 2 , … , n ; \qquad ( 14 )$$

S057, taking the belief factor δᵢ between the evidences as a weight, performing weighted averaging on an initial basic probability assignment, and defining a corrected basic probability assignment as follows:

$${m}^{*} ( A ) = {\sum }_{i = 1}^{n} {δ}_{i} \times {m}_{j} ( i ) ; \qquad ( 15 )$$

where A denotes a proposition in the evidence theory.

In some embodiments, the improving a combination rule of the D-S evidence theory based on a principle of local conflict distribution, fusing the corrected basic probability assignments by using an improved combination rule of the D-S evidence theory, and obtaining basic probability assignments of fatigue crack propagation of the steel structure at different development stages in S06 specifically include:
S061, computing a conflict distribution coefficient:

$$ε = \frac{{δ}_{i} {m}_{i}^{*} ( {A}_{i} )}{{δ}_{i} {m}_{i}^{*} ( {A}_{i} ) + {δ}_{j} {m}_{j}^{*} ( {A}_{j} ) + ⋯} ; \qquad ( 16 )$$

where m*(Aᵢ) denotes a corrected basic probability assignment of a proposition Aᵢ, m*(Aⱼ) denotes a corrected basic probability assignment of a proposition Aⱼ, δᵢ denotes a belief factor of an evidence in the proposition Aᵢ, δⱼ denotes a belief factor of an evidence in the proposition Aⱼ, and ε denotes the conflict distribution coefficient; and
S062, fusing corrected evidence sources by using the improved combination rule of the D-S evidence theory, where the combination rule is as follows:

$$\begin{matrix}m ( A ) = {\sum }_{\begin{matrix}{A}_{i} ⋂ {A}_{j} ⋂ ⋯ = A \\ {A}_{i} , {A}_{j} , ⋯ ⊆ θ\end{matrix}} {m}_{1}^{*} ( {A}_{i} ) \cdot {m}_{2}^{*} ( {A}_{j} ) \cdot … + f ( A ) ; & ( 17 ) \\ f ( A ) = {\sum }_{\begin{matrix}{A}_{i} ⋂ {A}_{j} ⋂ ⋯ = \\ {A}_{i} , {A}_{j} , ⋯ ⊆ θ\end{matrix}} ε [ {m}_{i}^{*} ( {A}_{i} ) \cdot {m}_{j}^{*} ( {A}_{j} ) \cdot … ] ; & ( 18 )\end{matrix}$$

where m(A) denotes a basic probability assignment of a proposition A, f(A) denotes a sum of conflict focal elements assigned to the proposition A; ε denotes the conflict distribution coefficient and determines conflict magnitudes assigned to the propositions, Aᵢ and Aⱼ denote corresponding propositions, Φ denotes a group of empty sets, and θ denotes a frame of discernment of the proposition, θ={A₁, A₂, . . . }.

In some embodiments, the multi-factor coupling cooperative early warning method further includes S063:
obtaining basic probability assignments m(A₁), m(A₂), m(A₃) and m(A₄) of the fatigue crack propagation of the steel structure at a crack initiation stage, a low-rate propagation stage, a high-rate propagation stage and an unstable propagation stage, where A₁ denotes the crack initiation stage, A₂ denotes the low-rate propagation stage, A₃ denotes the high-rate propagation stage and A₄ denotes the unstable propagation stage.

In some embodiments, the determining a fatigue crack propagation grade of the dangerous source distribution point of the steel structure project by a decision-making method for the basic probability assignment, and implementing time-varying prediction, a stage-based early warning and a probability early warning of the fatigue crack propagation process of the steel structure in S07 satisfy as follows:

$$m ( {ω}_{1} ) = max \{ m ( {ω}_{i} ) , {ω}_{i} ⊂ θ \} \\ m ( {ω}_{2} ) = max \{ m ( {ω}_{i} ) , {ω}_{i} ⊂ θ and {ω}_{i} \neq {ω}_{1} \} \\ \{ \begin{matrix}m ( {ω}_{1} ) - m ( {ω}_{2} ) > {λ}_{1} \\ m ( θ ) < {λ}_{2} \\ m ( {ω}_{1} ) > m ( θ )\end{matrix} ; \qquad ( 19 )$$

where m(ω₁) denotes a basic probability assignment of a proposition ω₁, ωᵢ denotes the proposition, θ denotes a frame of discernment of the proposition, m(ω₂) denotes a basic probability assignment of a proposition ω₂, m(θ) denotes a basic probability assignment returning to the frame of discernment θ, and λ₁ and λ₂ denote a set first threshold and a second threshold respectively; and under the condition that the formula (19) is satisfied, ω₁ denotes a final evaluation result, and m(ω₁) denotes the fatigue crack propagation grade of the dangerous source distribution point of the steel structure project.

The present disclosure further provides a multi-factor coupling cooperative early warning system for fatigue crack propagation of a steel structure. The multi-factor coupling cooperative early warning system is configured to execute the foregoing multi-factor coupling cooperative early warning method.

Compared with the prior art, the present disclosure has the beneficial effects: the multi-factor coupling cooperative early warning method and system for fatigue crack propagation of a steel structure according to the present disclosure solve the problem of a large error in a prediction result of a single response index during the fatigue crack propagation of the steel structure, and enhance a role of a multi-physical field monitoring means in the fatigue crack propagation of the steel structure.

According to the present disclosure, a Minkowski distance formula is introduced for quantitatively characterizing the distance between the evidences. In this way, the belief between the evidences is described, and the weight for distributing the evidences is obtained. The evidences are preprocessed through weighted averaging, and influence of a conflicting evidence on a fusion result is reduced accordingly.

According to the present disclosure, the combination rule of the D-S evidence theory is improved by using the principle of local conflict distribution, and a corresponding conflict distribution coefficient is assigned to a focal element that causes a conflict between the evidences. In this way, refined distribution of the conflict is implemented, and under the condition that the evidences have a high conflict, an uncertainty of the fusion result is greatly reduced, and the fusion result is more reasonable and accurate.

According to the present disclosure, the corrected evidence sources are fused by using the improved combination rule of the D-S evidence theory, the basic probability assignments of the fatigue crack propagation of the steel structure at different development stages are obtained. An evaluation result of the fatigue crack propagation of the steel structure is expressed with probabilities of different risk grades, the grade and the probability of the fatigue crack of the steel structure are intuitively reflected, and the time-varying prediction, the stage-based early warning and the probability early warning during the fatigue crack propagation of the steel structure are implemented.

Compared with the traditional D-S evidence theory, a data fusion algorithm used by the present disclosure is suitable for a case where the evidences support one another, and can further process conflict information of the evidences well. In this way, the uncertainty of the fusion result is greatly reduced and the fusion result is made more reasonable and accurate.

### BRIEF DESCRIPTION OF THE DRAWINGS

In order to describe an implementation manner of the present disclosure or a technical solution in the prior art more clearly, accompanying drawings required by the description of the implementation manner or the prior art will be briefly introduced below. Apparently, the accompanying drawings in the following description are merely illustrative, and a person of ordinary skill in the art can still derive other accompanying drawings from the accompanying drawings provided herein without creative efforts.

The structure, scale, size, etc. drawn and shown in the description are merely used to match the contents disclosed in the description and to be reading and understanding by those skilled in the art rather than to limit implementable limitation conditions of the present disclosure and are not technically substantive accordingly. Any structural modification, scale change, or size adjustment made without affecting the effects and objectives that can be achieved by the present disclosure shall fall within the scope covered by the technical content of the present disclosure.

FIG. 1 is a flowchart of a multi-factor coupling cooperative early warning method for fatigue crack propagation of a steel structure according to the present disclosure; and

FIG. 2 is a schematic diagram showing an early warning result of fatigue crack propagation of a steel structure according to an embodiment of the present disclosure.

### DETAILED DESCRIPTION OF THE EMBODIMENTS

To make objectives, technical solutions, and advantages of embodiments of the present disclosure clearer, the embodiments of the present disclosure will be further described in detail with reference to the embodiments and accompanying drawings. Illustrative embodiments and their description of the present disclosure are intended to explain the present disclosure herein, rather than to limit the present disclosure.

In the present disclosure, unless otherwise clearly specified, the terms such as “mount”, “connected”, “connection” and “fix” should be understood broadly. For example, a connection can be a fixed connection, a detachable connection or an integrated connection, can be a mechanical connection or an electric connection, can be a direct connection or an indirect connection through an intermediate medium, and can be internal communication of two elements or interaction between two elements. Those of ordinary skill in the art can understand specific meanings of the above terms in the present disclosure based on a specific situation.

It should be further understood that the terms “comprise/include”, “composed of”, or their any other variations are intended to cover non-exclusive including, such that a product, an apparatus, a process, or a method that includes a series of elements not only includes those elements, but also includes other elements that are not explicitly listed, or also includes inherent elements of the product, the apparatus, the process, or the method. Where there are no more restrictions, elements defined by the statement “comprise/include . . . ” or “composed of . . . ” do not exclude other identical elements from the product, the apparatus, the process, or the method that includes the elements.

It should be also understood that orientations or position relations indicated by the terms such as “upper”, “lower”, “front”, “back”, “left”, “right”, “top”, “bottom”, “inner”, and “outer” are orientations or position relations shown in the accompanying drawings. These terms are merely used to facilitate description of the present disclosure and simplify the description, rather than to indicate or imply that a device, component, or structure that is referred to should have a specific orientation or constructed and operated in a specific orientation, and these terms cannot be understood as limitation to the present disclosure accordingly.

In addition, the terms “first” and “second” are merely used for a purpose of description, and should not be understood as an indication or implication of relative importance or implicit indication of a quantity of indicated technical features. Thus, features defined with “first” and “second” can explicitly or implicitly include one or more of the features. In the description of the present disclosure, “plurality of” means two or more, unless otherwise specifically limited explicitly.

An evidence theory in the present disclosure is established by famous scholars Dempster and Shafer, and is also referred to as a D-S evidence theory. The evidence theory mainly treats and analyzes a proposition by transforming same into a mathematical set. Since the set may include a plurality of elements, the evidence theory can better express an uncertainty of the proposition just on account of its fuzziness, which is different from a probability theory that merely considers a single element. In fact, the evidence theory is more like simulation of a normal way of thinking of human beings. A first problem is to observe and collect information, that is, evidences. Then determination is performed by synthesizing all aspects of information, and a final result of the problem is obtained, that is, evidence synthesis. The most important thing is to determine an answer range of the question (frame of discernment), a probability corresponding to evidence set distribution (basic belief distribution function) and combination of evidence probability data (Dempster combination rule).

The implementation of the present disclosure will be described in detail below with reference to preferred implementation manners.

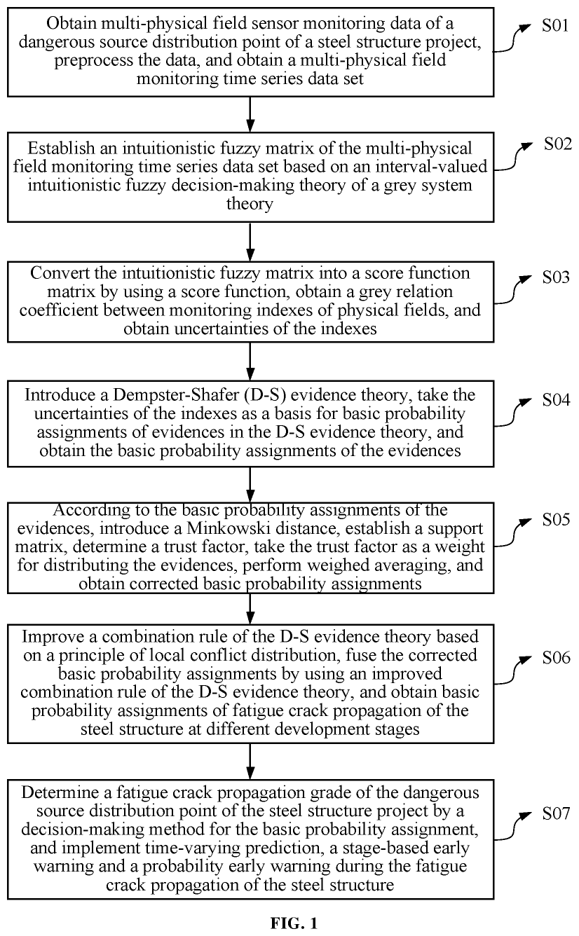

As shown in FIG. 1, a multi-factor coupling cooperative early warning method for fatigue crack propagation of a steel structure is provided according to the present disclosure. The multi-factor coupling cooperative early warning method specifically includes:
S01, obtain multi-physical field sensor monitoring data of a dangerous source distribution point of a steel structure project, preprocess the data, and obtain a multi-physical field monitoring time series data set.

The multi-physical field sensor monitoring data are real-time monitoring data collected by two or more sensors among a strain sensor, a displacement sensor, a stress sensor, a wave velocity sensor, a temperature sensor, an acoustic emission sensor and an electromagnetic radiation sensor according to a time series.

Multi-physical field monitoring time series data may be real-time monitoring data of a combination of two or more of monitoring indexes that include a displacement, a strain, a stress, a wave velocity, an osmotic pressure, a temperature, acoustic emission, electromagnetic radiation, etc.

In this embodiment, the multi-physical field sensor monitoring data include a crack length, a number of times of acoustic emission ringing and accumulated acoustic emission energy. A fatigue crack propagation test is carried out on concrete steel members of a steel structure. Original monitoring data are obtained by an acoustic emission monitoring system, and the multi-physical field monitoring time series data set are obtained.

In the present disclosure, the monitoring index refers to a name or a type of monitoring data, and the monitoring data are real-time data measured by sensors. For example, the number of times of acoustic emission ringing and the accumulated acoustic emission energy are two monitoring indexes obtained by a sensor of an acoustic emission apparatus.

S02, obtain an interval-valued intuitionistic fuzzy number according to the multi-physical field monitoring time series data set and by an interval-valued intuitionistic fuzzy decision-making method based on the grey system theory and establish an intuitionistic fuzzy matrix of the multi-physical field monitoring time series data set.

The step of establishing an intuitionistic fuzzy matrix basically includes:
S021, obtain an interval-valued intuitionistic fuzzy number according to the multi-physical field monitoring time series data set and by an interval-valued intuitionistic fuzzy decision-making method based on the grey system theory:

$${e}_{ij} = 〈 u ( x ) , v ( x ) 〉 ; \qquad ( 1 )$$

where u(x) and v(x) denote a membership degree and a non-membership degree of an element x, belonging to a fatigue crack development stage of the steel structure, in a monitoring index μⱼ respectively, j=1, 2, . . . , m; i=1, 2, . . . , n; and
S022, establish the intuitionistic fuzzy matrix, and establish an intuitionistic fuzzy decision-making matrix according to a monitoring index and a development stage of the fatigue crack propagation of the steel structure;

$$E = {( {e}_{ij} )}_{m \times n} ; \qquad ( 2 )$$

where eᵢⱼ denotes an attribute value under the monitoring index μⱼ of the fatigue crack development stage of the steel structure, and is referred to as the interval-valued intuitionistic fuzzy number.

S03, convert the intuitionistic fuzzy matrix into a score function matrix by using a score function, obtain and compute a grey relation coefficient between monitoring indexes of physical fields, and obtain uncertainties of the indexes.

In this embodiment, the fatigue crack propagation of the steel structure is a gradual process. In order to truly reflect the development stage of the crack propagation, the grey relation coefficient is introduced for characterizing the reliability of the development stage of the crack propagation.
S03 specifically includes: S031, define a score function:

$$g ( x ) = u ( x ) - v ( x ) ; \qquad ( 3 )$$

where g(x)∈[−1,1], g(x) expresses a difference between a support degree and an opposition degree, and denotes a net support degree, g(x)=−1 indicates absolute opposition, and g(x)=1 indicates absolute support;
S032, obtain a score matrix according to the score function:

$$G = {( {g}_{ij} )}_{m \times n} ; \qquad ( 4 )$$

S033, compute the grey relation coefficient between the indexes according to the score matrix:

$${r}_{ij} = \frac{min \vert {g}_{ij} - \overline{{g}_{i}} \vert + ρ max \vert {g}_{ij} - \overline{{g}_{i}} \vert}{\vert {g}_{ij} - \overline{{g}_{i}} \vert + ρ max \vert {g}_{ij} - \overline{{g}_{i}} \vert} , i = 1 , 2 , … , j = 1 , 2 , … ; \qquad ( 5 )$$

where rᵢⱼ denotes the grey relation coefficient between the indexes, gᵢⱼ denotes a score function value, g̅ᵢ denotes an average score function value, p denotes a weight coefficient and ρ=0.5 is generally set; and
S034, obtain the uncertainty of the monitoring index by using the grey relation coefficient;

$$DOI ( {μ}_{j} ) = {\frac{1}{m} [ {\sum }_{i = 1}^{m} {( {r}_{ij} )}^{q} ]}^{\frac{1}{q}} ; \qquad ( 6 )$$

where μⱼ denotes the monitoring index, m denotes a number of the monitoring indexes, rᵢⱼ denotes the grey relation coefficient between the indexes, q denotes a distance measurement coefficient, an Euclidean distance is used herein and q=2 is set.

S04, introduce a Dempster-Shafer (D-S) evidence theory, take the uncertainties of the indexes as bases for basic probability assignments of evidences in the D-S evidence theory, and obtain the basic probability assignments of the evidences.

Specifically, the uncertainties DOI(μⱼ) are taken as basic probability assignments of the monitoring indexes at different development stages:

$${m}_{j}^{*} ( i ) = \frac{( 1 - DOI ( {μ}_{j} ) ) \cdot {r}_{ij}}{{\sum }_{i = 1}^{m} {r}_{ij}} ; \qquad ( 7 )$$

Further, m*ⱼ(i) is corrected:

$${m}_{j} ( i ) = \frac{{m}_{j}^{*} ( i )}{{\sum }_{i = 1}^{m} {m}_{j}^{*} ( i )} ; \qquad ( 8 )$$

where an obtained mⱼ(i) denotes the basic probability assignment at different development stages under the monitoring index μⱼ.

S05, according to the basic probability assignments of the evidences, introduce a Minkowski distance, establish a support degree matrix, determine a belief factor, distribute the evidences, perform weighed averaging, and obtain corrected basic probability assignments.

A Minkowski distance formula is introduced for quantitatively characterizing the distance between the evidences. In this way, the belief between the evidences is described, and the weight for distributing the evidences is obtained. The evidences are preprocessed through weighted averaging, and influence of a conflicting evidence on a fusion result is reduced accordingly.

S05 specifically includes: S051, define a Minkowski distance between the evidences, where a Minkowski distance between n-dimensional vectors a(x₁₁, x₁₂ . . . , x₁ₙ) and b(x₂₁, x₂₂ . . . , x₂ₙ) is expressed as follows:

$${d}_{12} = \sqrt[p]{{\sum }_{k = 1}^{n} {( \vert {x}_{1 k} - {x}_{2 k} \vert )}^{p}} ; \qquad ( 9 )$$

where a and b are two n-dimensional vectors, x₁ₖ denotes a value of a vector at a first row and a k^{th} column, x₂ₖ denotes a value of a vector at a second row and a k^{th} column, P denotes a Minkowski index, p≥1 and PN⊂N, in the case that p=1, Formula (9) is a Taxicab distance; in the case that p=2, Formula (9) is an Euclidean distance; in the case that p=∞, Formula (9) is a Chebyshev distance.

The Minkowski distance is a common method for measuring the distance between numerical points. As the p changes, the Minkowski distance is expressed as different distances, and the Taxicab distance, the Euclidean distance and the Chebyshev distance are all special cases of Minkowski distance. The present disclosure may select the most suitable Minkowski distance according to data features of a specific sample.

S052, compute the Minkowski distance between the evidences by using formula (9) and obtain a distance matrix dᵢⱼ as follows:

$${d}_{ij} = [ \begin{matrix}0 & & {d}_{12} & & {d}_{1 n} \\ & & & ⋯ & \\ {d}_{21} & & 0 & & {d}_{2 n} \\ & ⋮ & & ⋱ & ⋮ \\ {d}_{n 1} & {d}_{n 2} & ⋯ & & 0\end{matrix} ] ; \qquad ( 10 )$$

S053, quantitatively characterize a support degree between the evidences through the distance matrix, and define the support degree supᵢⱼ between the evidences as follows:

$${sup}_{ij} = {e}^{- {d}_{ij}} ; \qquad ( 11 )$$

The smaller the Minkowski distance between two evidences is, the greater the support degree of the evidences is, and the higher the belief between the evidences is.

S054, obtain the support degree matrix S according to the support degree:

$$S = [ \begin{matrix}1 & {sup}_{12} & {sup}_{13} & & {sup}_{1 n} \\ & & & ⋯ & \\ {sup}_{21} & 1 & {sup}_{23} & & {sup}_{2 n} \\ & ⋮ & & ⋱ & ⋮ \\ {sup}_{n 1} & {sup}_{n 2} & {sup}_{n 3} & ⋯ & 1\end{matrix} ] ; \qquad ( 12 )$$

S055, sum all elements except a designated element in each row of the support degree matrix and obtain an inter-evidence support degree recᵢ:

$${rec}_{i} = {\sum }_{j = 1 , i \neq j}^{n} {sup}_{ij} , i , j = 1 , 2 , … , n ; \qquad ( 13 )$$

In the present disclosure, the greater the recᵢ is, the higher the support degree between the evidences is. The smaller the recᵢ is, the lower the support degree between evidences is.

S056, measure the support degree between the evidences by using the belief factor, and take the support degree as a weight for distributing a conflict probability, where the belief factor δᵢ between the evidences is as follows:

$${δ}_{i} = \frac{{rec}_{i}}{{\sum }_{i = 1}^{n} {rec}_{i}} , i = 1 , 2 , … , n ; \qquad ( 14 )$$

S057, take the belief factor δᵢ between the evidences as a weight, perform weighted averaging on an initial basic probability assignment, and define a corrected basic probability assignment as follows:

$${m}^{*} ( A ) = {\sum }_{i = 1}^{n} {δ}_{i} \times {m}_{j} ( i ) ; \qquad ( 15 )$$

where A denotes a proposition in the evidence theory, and refers to a development stage of the steel structure, that is, the following four development stages.

An initial basic probability assignment in the present disclosure is the basic probability assignment obtained through S04 of the monitoring indexes at different development stages.

S06, improve a combination rule of the D-S evidence theory based on a principle of local conflict distribution, fuse the corrected basic probability assignments by using an improved combination rule of the D-S evidence theory, and obtain basic probability assignments of fatigue crack propagation of the steel structure at different development stages.

According to the present disclosure, the combination rule of the D-S evidence theory is improved by using a method of local conflict distribution, and a corresponding conflict distribution coefficient is assigned to a focal element that causes a conflict between the evidences. In this way, refined distribution of the conflict is implemented, and under the condition that the evidences have a high conflict, an uncertainty of the fusion result is greatly reduced, and the fusion result is more reasonable and accurate.

S06 specifically includes: S061, compute a conflict distribution coefficient:

$$ε = \frac{{δ}_{i} {m}_{i}^{*} ( {A}_{i} )}{{δ}_{i} {m}_{i}^{*} ( {A}_{i} ) + {δ}_{j} {m}_{j}^{*} ( {A}_{j} ) + ⋯} ; \qquad ( 16 )$$

where m*(Aᵢ) denotes a corrected basic probability assignment of a proposition Aᵢ, m*(Aⱼ) denotes a corrected basic probability assignment of a proposition Aⱼ, δᵢ denotes a belief factor of an evidence in the proposition Aᵢ, δⱼ denotes a belief factor of an evidence in the proposition Aⱼ, and ε denotes the conflict distribution coefficient.

S062, fuse corrected evidence sources by using the improved combination rule of the D-S evidence theory, where the combination rule is as follows:

$$\begin{matrix}m ( A ) = {\sum }_{\begin{matrix}{A}_{i} ⋂ {A}_{j} ⋂ ⋯ = A \\ {A}_{i} , {A}_{j} , ⋯ ⊆ θ\end{matrix}} {m}_{1}^{*} ( {A}_{i} ) \cdot {m}_{2}^{*} ( {A}_{j} ) \cdot … + f ( A ) & ( 17 ) \\ f ( A ) = {\sum }_{\begin{matrix}{A}_{i} ⋂ {A}_{j} ⋂ ⋯ = Φ \\ {A}_{i} , {A}_{j} , ⋯ ⊆ θ\end{matrix}} ε [ {m}_{i}^{*} ( {A}_{i} ) \cdot {m}_{j}^{*} ( {A}_{j} ) \cdot … ] ; & ( 18 )\end{matrix}$$

where m(A) denotes a basic probability assignment of a proposition A, f(A) denotes a sum of conflict focal elements assigned to the proposition A; ε denotes the conflict distribution coefficient and determines conflict magnitudes assigned to the propositions, Aᵢ and Aⱼ denote corresponding propositions, Φ denotes a group of empty sets, and θ denotes a frame of discernment of the proposition, θ={A₁, A₂, . . . }.

S063, obtain basic probability assignments m(A₁), m(A₂), m(A₃) and m(A₄) of the fatigue crack propagation of the steel structure at a crack initiation stage, a low-rate propagation stage, a high-rate propagation stage and an unstable propagation stage, where A₁ denotes the crack initiation stage, A₂ denotes the low-rate propagation stage, A₃ denotes the high-rate propagation stage and A₄ denotes the unstable propagation stage.

In this step, based on the corrected evidence source, the multi-physical field monitoring data of the fatigue crack propagation of the steel structure are fused according to the improved combination rule of the D-S evidence theory.

S07, determine a fatigue crack propagation grade of the dangerous source distribution point of the steel structure project by a decision-making method for the basic probability assignment, and implement time-varying prediction, a stage-based early warning and a probability early warning during the fatigue crack propagation of the steel structure.

In this step, the decision-making method of the basic probability assignment is used for evaluating the fatigue crack propagation grade of the steel structure as follows:

$$m ( {ω}_{1} ) = max \{ m ( {ω}_{i} ) , {ω}_{i} ⊂ θ \} , \\ m ( {ω}_{2} ) = max \{ m ( {ω}_{i} ) , {ω}_{i} ⊂ θ and {ω}_{i} \neq {ω}_{1} \} \\ \{ \begin{matrix}m ( {ω}_{1} ) - m ( {ω}_{2} ) > {λ}_{1} \\ m ( θ ) < {λ}_{2} \\ m ( {ω}_{1} ) > m ( θ )\end{matrix} ; \qquad ( 19 )$$

where m(ω₁) denotes a basic probability assignment of a proposition ω₁, ωᵢ denotes the proposition, θ denotes a frame of discernment of the proposition, m(ω₂) denotes a basic probability assignment of a proposition ω₂, ε₁ and ε₂ denote thresholds, thresholds ε₁=ε₂=0.1 may be set; m(θ) denotes a basic probability assignment returning to the frame of discernment θ, and λ₁ and λ₂ denote a set first threshold and a second threshold respectively, λ₁=λ₂=0.01 is regularly set; and under the condition that the formula (19) is satisfied, ω1 denotes a final evaluation result, and m(ω1) denotes the fatigue crack propagation grade of the dangerous source distribution point of the steel structure project.

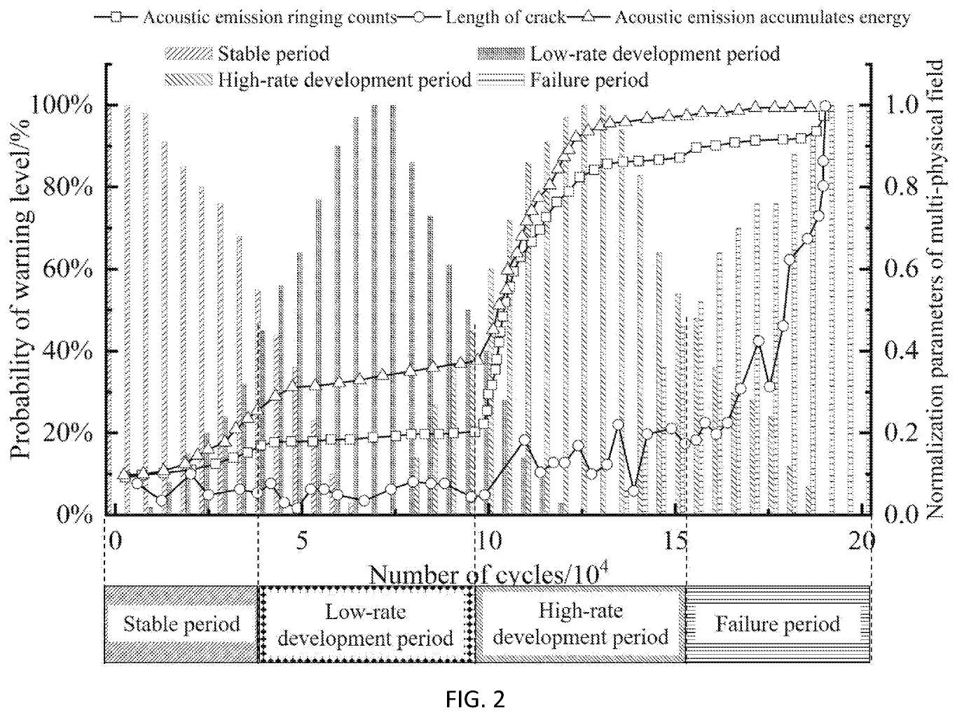

In the embodiment of the present disclosure, the fatigue crack propagation grade of the dangerous source distribution point of the steel structure project is determined by the method for the basic probability assignment, and an early warning result of the fatigue crack propagation of the steel structure is shown in FIG. 2. In FIG. 2, a left vertical axis denotes an early warning grade probability, and a right vertical axis denotes a multi-physical field normalized parameter. In this embodiment, the multi-physical field monitoring data mainly include the number of times of acoustic emission ringing, the crack length and the accumulated acoustic emission energy. Numerical values on a column diagram correspond to early warning probabilities of the dangerous source distribution point of the steel structure at different numbers of cycles. Decision-making results of the basic probability assignment method are below a horizontal axis, and correspond to a stable period (crack initiation stage), a low-rate development period (low-rate crack propagation stage) and a high-rate development period (high-rate crack propagation stage) from left to right. In this diagram, the early warning grade probabilities of the dangerous source distribution point of the steel structure at different development stages can be obtained at different numbers of cycles. An evaluation result of the fatigue crack propagation of the steel structure is expressed with probabilities of different risk grades, the grade and the probability of the fatigue crack of the steel structure are intuitively reflected, and the time-varying prediction, the stage-based early warning and the probability early warning during the fatigue crack propagation of the steel structure are implemented.

The present disclosure further provides a multi-factor coupling cooperative early warning system for fatigue crack propagation of a steel structure for executing the multi-factor coupling cooperative early warning method. The multi-factor coupling cooperative early warning system can be implemented by a software program, an early warning can be given more efficiently and quickly and early warning efficiency is improved accordingly.

The present disclosure solves the problem of a large error in a prediction result of a single response index during the fatigue crack propagation of the steel structure, and enhances a role of a multi-physical field monitoring means in the fatigue crack propagation of the steel structure.

Those skilled in the art can easily understand that the preferred solutions can be freely combined and superimposed without conflict.

The above implementation manners are merely preferred implementation manners of the present disclosure, and not intended to limit the present disclosure. Any modification, equivalent replacement, improvement, etc. made without departing from the spirit and principle of the present disclosure should fall within the protection scope of the present disclosure.

What is claimed is:

1. A multi-factor coupling cooperative early warning method for fatigue crack propagation of a steel structure, comprising the following steps:
S01, obtaining multi-physical field sensor monitoring data of a dangerous source distribution point of a steel structure project, preprocessing the data, and obtaining a multi-physical field monitoring time series data set;
S02, establishing an intuitionistic fuzzy matrix of the multi-physical field monitoring time series data set based on an interval-valued intuitionistic fuzzy decision-making theory of a grey system theory;
S03, converting the intuitionistic fuzzy matrix into a score function matrix by using a score function, obtaining a grey relation coefficient between monitoring indexes of physical fields, and obtaining uncertainties of the indexes;
S04, introducing a Dempster-Shafer (D-S) evidence theory, taking the uncertainties of the indexes as bases for basic probability assignments of evidences in the D-S evidence theory, and obtaining the basic probability assignments of the evidences;
S05, according to the basic probability assignments of the evidences, introducing a Minkowski distance, establishing a support degree matrix, determining a belief factor, taking the belief factor as a weight for distributing the evidences, performing weighed averaging, and obtaining corrected basic probability assignments;
S06, improving a combination rule of the D-S evidence theory based on a principle of local conflict distribution, fusing the corrected basic probability assignments by using an improved combination rule of the D-S evidence theory, and obtaining basic probability assignments of fatigue crack propagation of the steel structure at different development stages; and
S07, determining a fatigue crack propagation grade of the dangerous source distribution point of the steel structure project by a decision-making method for the basic probability assignment, and implementing time-varying prediction, a stage-based early warning and a probability early warning during the fatigue crack propagation of the steel structure.

2. The multi-factor coupling cooperative early warning method according to claim 1, wherein the obtaining multi-physical field sensor monitoring data of a dangerous source distribution point of a steel structure project, preprocessing the data, and obtaining a multi-physical field monitoring time series data set in S01 specifically comprise:
obtaining multi-sensor real-time monitoring data of the dangerous source distribution point of the steel structure project, wherein the multi-physical field sensor monitoring data are real-time monitoring data collected by two or more sensors among a strain sensor, a displacement sensor, a stress sensor, a wave velocity sensor, a temperature sensor, an acoustic emission sensor and an electromagnetic radiation sensor according to a time series; and
multi-physical field monitoring time series data are real-time monitoring data of a combination of two or more of monitoring indexes that comprise a displacement, a strain, a stress, a wave velocity, an osmotic pressure, a temperature, acoustic emission and electromagnetic radiation.

3. The multi-factor coupling cooperative early warning method according to claim 1,
wherein the establishing an intuitionistic fuzzy matrix of the multi-physical field monitoring time series data set in S02 specifically comprises:
S021, obtaining an interval-valued intuitionistic fuzzy number according to the multi-physical field monitoring time series data set and by an interval-valued intuitionistic fuzzy decision-making method based on the grey system theory:
$${e}_{ij} = 〈 u ( x ) , v ( x ) 〉 ; \qquad ( 1 )$$
wherein u(x) and v(x) denote a membership degree and a non-membership degree of an element x, belonging to a fatigue crack development stage of the steel structure, in a monitoring index μⱼ respectively, j=1, 2, . . . , m; i=1, 2, . . . , n; and
S022, establishing the intuitionistic fuzzy matrix, and establishing an intuitionistic fuzzy decision-making matrix according to a monitoring index and a development stage of the fatigue crack propagation of the steel structure;
$$E = {( {e}_{ij} )}_{m \times n} ; \qquad ( 2 )$$
wherein eᵢⱼ denotes an attribute value under the monitoring index μⱼ of the fatigue crack development stage of the steel structure, and is referred to as the interval-valued intuitionistic fuzzy number.

4. The multi-factor coupling cooperative early warning method according to claim 1, wherein the converting the intuitionistic fuzzy matrix into a score function matrix by using a score function, obtaining a grey relation coefficient between monitoring indexes of physical fields, and obtaining uncertainties of the indexes in S03 specifically comprise:
S031, defining the score function:
$$g ( x ) = u ( x ) - v ( x ) ; \qquad ( 3 )$$
wherein g(x)∈[−1,1], g(x) expresses a difference between a support degree and an opposition degree, and denotes a net support degree, g(x)=−1 indicates absolute opposition, and g(x)=1 indicates absolute support;
S032, obtaining a score matrix according to the score function:
$$G = {( {g}_{ij} )}_{m \times n} ; \qquad ( 4 )$$
S033, computing the grey relation coefficient between the indexes according to the score matrix:
$${r}_{ij} = \frac{min \vert {g}_{ij} - \overline{{g}_{i}} \vert + ρ max \vert {g}_{ij} - \overline{{g}_{i}} \vert}{\vert {g}_{ij} - \overline{{g}_{i}} \vert + ρ max \vert {g}_{ij} - \overline{{g}_{i}} \vert} , i = 1 , 2 , … , j = 1 , 2 , … ; \qquad ( 5 )$$
wherein rᵢⱼ denotes the grey relation coefficient between the indexes, gᵢⱼ denotes a score function value, g̅ᵢ denotes an average score function value, and ρ denotes a weight coefficient; and
S034, obtaining the uncertainty of the monitoring index by using the grey relation coefficient;
$$DOI ( {μ}_{j} ) = {\frac{1}{m} [ {\sum }_{i = 1}^{m} {( {r}_{ij} )}^{q} ]}^{\frac{1}{q}} ; \qquad ( 6 )$$
wherein μⱼ denotes the monitoring index, m denotes a number of the monitoring indexes, rᵢⱼ denotes the grey relation coefficient between the indexes, and q denotes a distance measurement coefficient.

5. The multi-factor coupling cooperative early warning method according to claim 4, wherein the obtaining basic probability assignments of the monitoring indexes at different development stages by using the uncertainty DOI(μⱼ) in S04 comprises:
$${m}_{j}^{*} ( i ) = \frac{( 1 - DOI ( {μ}_{j} ) ) \cdot {r}_{ij}}{{\sum }_{i = 1}^{m} {r}_{ij}} ; \qquad ( 7 )$$
and
correcting m*ⱼ(i):
$${m}_{j} ( i ) = \frac{{m}_{j}^{*} ( i )}{{\sum }_{i = 1}^{m} {m}_{j}^{*} ( i )} ; \qquad ( 8 )$$
wherein an obtained mⱼ(i) denotes the basic probability assignment at different development stages under the monitoring index μⱼ.

6. The multi-factor coupling cooperative early warning method according to claim 1, wherein the introducing a Minkowski distance, establishing a support degree matrix, determining a belief factor, taking the belief factor as a weight for distributing the evidences, performing weighed averaging, performing weighted averaging, and obtaining a corrected basic probability assignment in S05 specifically comprise:
S051, defining a Minkowski distance between the evidences, wherein a Minkowski distance between n-dimensional vectors a (x₁₁, x₁₂, . . . , x₁ₙ) and b(x₂₁, x₂₂, . . . , x₂ₙ) is expressed as follows:
$${d}_{12} = \sqrt[p]{{\sum }_{k = 1}^{n} {( \vert {x}_{1 k} - {x}_{2 k} \vert )}^{p}} \qquad ( 9 )$$
wherein a and b are two n-dimensional vectors, x₁ₖ denotes a value of a vector at a first row and a k^{th} column, x₂ₖ denotes a value of a vector at a second row and a k^{th} column, P denotes a Minkowski index, p≥1 and PN⊂N;
S052, computing the Minkowski distance between the evidences by using formula (9) and obtaining a distance matrix dᵢⱼ as follows:
$${d}_{ij} = [ \begin{matrix}0 & & {d}_{12} & & {d}_{1 n} \\ & & & ⋯ & \\ {d}_{21} & & 0 & & {d}_{2 n} \\ & ⋮ & & ⋱ & ⋮ \\ {d}_{n 1} & & {d}_{n 2} & ⋯ & 0\end{matrix} ] ; \qquad ( 10 )$$
S053, quantitatively characterizing a support degree between the evidences through the distance matrix, and defining the support degree supᵢⱼ between the evidences as follows:
$${sup}_{ij} = {e}^{- {d}_{ij}} ; \qquad ( 11 )$$
S054, obtaining the support degree matrix S according to the support degree:
$$S = [ \begin{matrix}1 & {sup}_{12} & {sup}_{13} & & {sup}_{1 n} \\ & & & ⋯ & \\ {sup}_{21} & 1 & {sup}_{23} & & {sup}_{2 n} \\ & ⋮ & & ⋱ & ⋮ \\ {sup}_{n 1} & {sup}_{n 2} & {sup}_{n 3} & & 1\end{matrix} ] ; \qquad ( 12 )$$
S055, summing all elements except a designated element in each row of the support degree matrix and obtaining an inter-evidence support degree recᵢ:
$${rec}_{i} = {\sum }_{j = , i \neq j}^{n} {sup}_{ij} , i , j = 1 , 2 , … , n ; \qquad ( 13 )$$
S056, measuring the support degree between the evidences by using the belief factor, wherein the belief factor δᵢ between the evidences is as follows:
$${δ}_{i} = \frac{{rec}_{i}}{{\sum }_{i = 1}^{n} {rec}_{i}} , i = 1 , 2 , … , n ; \qquad ( 14 )$$
S057, taking the belief factor δᵢ between the evidences as a weight, performing weighted averaging on an initial basic probability assignment, and defining a corrected basic probability assignment as follows:
$${m}^{*} ( A ) = {\sum }_{i = 1}^{n} {δ}_{i} \times {m}_{j} ( i ) ; \qquad ( 15 )$$
wherein A denotes a proposition in the evidence theory.

7. The multi-factor coupling cooperative early warning method according to claim 1, wherein the improving a combination rule of the D-S evidence theory based on a principle of local conflict distribution, fusing the corrected basic probability assignments by using an improved combination rule of the D-S evidence theory, and obtaining basic probability assignments of fatigue crack propagation of the steel structure at different development stages in S06 specifically comprise:
S061, computing a conflict distribution coefficient:
$$ε = \frac{{δ}_{i} {m}_{i}^{*} ( {A}_{i} )}{{δ}_{i} {m}_{i}^{*} ( {A}_{i} ) + {δ}_{j} {m}_{j}^{*} ( {A}_{j} ) + ⋯} ; \qquad ( 16 )$$
wherein m*(Aᵢ) denotes a corrected basic probability assignment of a proposition Aᵢ, m*(Aⱼ) denotes a corrected basic probability assignment of a proposition Aⱼ, δᵢ denotes a belief factor of an evidence in the proposition Aᵢ, δⱼ denotes a belief factor of an evidence in the proposition Aⱼ, and ε denotes the conflict distribution coefficient; and
S062, fusing corrected evidence sources by using the improved combination rule of the D-S evidence theory, wherein the combination rule is as follows:
$$\begin{matrix}m ( A ) = {\sum }_{\begin{matrix}{A}_{i} ⋂ {A}_{j} ⋂ ⋯ = A \\ {A}_{i} , {A}_{j} , ⋯ ⊆ θ\end{matrix}} {m}_{1}^{*} ( {A}_{i} ) \cdot {m}_{2}^{*} ( {A}_{j} ) \cdot … + f ( A ) & ( 17 ) \\ f ( A ) = {\sum }_{\begin{matrix}{A}_{i} ⋂ {A}_{j} ⋂ ⋯ = Φ \\ {A}_{i} , {A}_{j} , ⋯ ⊆ θ\end{matrix}} ε [ {m}_{i}^{*} ( {A}_{i} ) \cdot {m}_{j}^{*} ( {A}_{j} ) \cdot … ] ; & ( 18 )\end{matrix}$$
wherein m(A) denotes a basic probability assignment of a proposition A, f(A) denotes a sum of conflict focal elements assigned to the proposition A; ε denotes the conflict distribution coefficient and determines conflict magnitudes assigned to the propositions, Ai and Aj denote corresponding propositions, Φ denotes a group of empty sets, and θ denotes a frame of discernment of the proposition, θ={A₁, A₂, . . . }.

8. The multi-factor coupling cooperative early warning method according to claim 7, further comprising S063:
obtaining basic probability assignments m(A₁), m(A₂), m(A₃) and m(A₄) of the fatigue crack propagation of the steel structure at a crack initiation stage, a low-rate propagation stage, a high-rate propagation stage and an unstable propagation stage, wherein A₁ denotes the crack initiation stage, A₂ denotes the low-rate propagation stage, A₃ denotes the high-rate propagation stage, and A₄ denotes the unstable propagation stage.

9. The multi-factor coupling cooperative early warning method according to claim 1, wherein the determining a fatigue crack propagation grade of the dangerous source distribution point of the steel structure project by a decision-making method for the basic probability assignment, and implementing time-varying prediction, a stage-based early warning and a probability early warning of the fatigue crack propagation process of the steel structure in S07 satisfy as follows:
$$m ( {ω}_{1} ) = max \{ m ( {ω}_{i} ) , {ω}_{i} ⊂ θ \} , \\ m ( {ω}_{2} ) = max \{ m ( {ω}_{i} ) , {ω}_{i} ⊂ θ and {ω}_{i} \neq {ω}_{1} \} \\ \{ \begin{matrix}m ( {ω}_{1} ) - m ( {ω}_{2} ) > {λ}_{1} \\ m ( θ ) < {λ}_{2} \\ m ( {ω}_{1} ) > m ( θ )\end{matrix} ; \qquad ( 19 )$$
wherein m(ω₁) denotes a basic probability assignment of a proposition ω₁, ωᵢ denotes the proposition, θ denotes a frame of discernment of the proposition, m(ω₂) denotes a basic probability assignment of a proposition ω₂, m(θ) denotes a basic probability assignment returning to the frame of discernment θ, and λ₁ and λ₂ denote a set first threshold and a second threshold respectively, and under the condition that the formula (19) is satisfied, ω₁ denotes a final evaluation result, and m (ω₁) denotes the fatigue crack propagation grade of the dangerous source distribution point of the steel structure project.

10. A multi-factor coupling cooperative early warning system for fatigue crack propagation of a steel structure, configured to execute the multi-factor coupling cooperative early warning method according to claim 1.

11. A multi-factor coupling cooperative early warning system for fatigue crack propagation of a steel structure, configured to execute the multi-factor coupling cooperative early warning method according to claim 2.

12. A multi-factor coupling cooperative early warning system for fatigue crack propagation of a steel structure, configured to execute the multi-factor coupling cooperative early warning method according to claim 3.

13. A multi-factor coupling cooperative early warning system for fatigue crack propagation of a steel structure, configured to execute the multi-factor coupling cooperative early warning method according to claim 4.

14. A multi-factor coupling cooperative early warning system for fatigue crack propagation of a steel structure, configured to execute the multi-factor coupling cooperative early warning method according to claim 5.

15. A multi-factor coupling cooperative early warning system for fatigue crack propagation of a steel structure, configured to execute the multi-factor coupling cooperative early warning method according to claim 6.

16. A multi-factor coupling cooperative early warning system for fatigue crack propagation of a steel structure, configured to execute the multi-factor coupling cooperative early warning method according to claim 7.

17. A multi-factor coupling cooperative early warning system for fatigue crack propagation of a steel structure, configured to execute the multi-factor coupling cooperative early warning method according to claim 8.

18. A multi-factor coupling cooperative early warning system for fatigue crack propagation of a steel structure, configured to execute the multi-factor coupling cooperative early warning method according to claim 9.
